(19) United States
(12) Patent Application Publication
(10) Pub. No.: US 2010/0047836 A1
(43) Pub. Date: Feb. 25, 2010

(54) LIPOLYTIC ENZYME VARIANTS

(75) Inventors: Leonardo De Maria, Frederiksberg (DK); Jesper Brask, Bagsvaerd (DK); Michael Skjot, Jyllinge (DK); Shamkant Anant Patkar, Lyngby (DK); Kim Borch, Birkerod (DK); Allan Svendsen, Hoersholm (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/515,686

(22) PCT Filed: Nov. 26, 2007
(86) PCT No.: PCT/EP07/62783

§ 371 Date: Jun. 17, 2009

Related U.S. Application Data
(60) Provisional application No. 60/861,306, filed on Nov. 28, 2006.

(30) Foreign Application Priority Data
Nov. 28, 2006 (DK) PA 2006 01560

(51) Int. Cl. C12Q 1/44; C12N 9/20

(52) U.S. Cl. 435/19; 435/198

(57) ABSTRACT

Molecular dynamics (MD) simulation on the three-dimensional structure of Candida anrtarctica lipase B revealed two hitherto unknown lids with a marked mobility, and this discovery was used to design lipolytic enzyme variants with increased lipolytic enzyme activity.

### FIELD OF THE INVENTION

The present invention relates to a polypeptide with lipolytic enzyme activity and to a method of preparing it.

### BACKGROUND OF THE INVENTION

WO8802775 describes Candida antarctica lipase B (CALB). Uppenberg, Hansen, Patkar, Jones, Structure 2, 293-308 (1994) describe the amino acid sequence and three-dimensional (3D) structure of CALB. The 3D structure can be found in the Research Collaboratory for Structural Bioinformatics Protein Data Bank (RCBS PDB) (http://www.rcsb.org/), its identifier being 1TCA.

CALB variants are described in Zhang et al. Prot. Eng. 2003, 16, 599-605; Lutz. 2004, Tetrahedron: Asymmetry, 15, 2743-2748; Qian and Lutz, JACS, 2005, 127, 13466-13467; and in WO 2004/024954.

WO9324619 describes a lipase from Hyphozyma sp. Amino acid sequences for other lipases can be found in UniProt [the Universal Protein Resource] with accession numbers Q4pep1, Q7RYD2, Q2UE03, Q4WG73, Q6BVP4 and Q4HUY1.

### SUMMARY OF THE INVENTION

The inventors performed molecular dynamics (MD) simulation on the 1TCA structure. The analysis reveals two hitherto unknown lids with a marked mobility, Lid 1 consisting of residues from 135 or 136 to 155 or 160, and Lid 2 consisting of residues 267-295. The simulation indicated a more closed like form in water solution and a more fully open form in organic solvent solution. The analysis revealed important areas in the 3D structure for affecting the activity and functionality of the lipase, and the inventors used this to design lipolytic enzyme variants with increased specific activity, particularly towards bulky substrates (e.g. esters of a branched acid or long-chain fatty acid and/or a secondary alcohol) and/or increased activity at high pH (higher pH optimum) and/or increased enantioselectivity.

Further, the inventors have selected amino acid residues and designed lipolytic enzyme variants based on an alignment of CALB with some homologous lipase sequences.

Accordingly, the invention provides a method of preparing a polypeptide, comprising

a) selecting a parent polypeptide which has lipolytic enzyme activity and has an amino acid sequence with at least 30% identity to CALB (SEQ ID NO: 1),

b) selecting one or more amino acid residues in the sequence corresponding to any of residues 1, 13, 25, 38-51, 53-55, 58, 69-79, 91, 92, 96, 97, 99, 103, 104-110, 113, 132-168, 173, 187-193, 197-205, 215, 223-231, 242, 244, 256, 259, 261-298, 303, 305, 308-313, or 315 of CALB (SEQ ID NO: 1),

c) altering the selected amino acid sequence wherein the alteration comprises substitution or deletion of the selected residue(s) or insertion of at least one residue adjacent to the selected residue(s),

d) preparing an altered polypeptide having the altered amino acid sequence,

e) determining the lipolytic enzyme activity or enantioselectivity towards carboxylic ester bonds of the altered polypeptide, and

f) selecting an altered polypeptide which has higher lipolytic enzyme activity or a higher enantioselectivity than the parent polypeptide.

The invention also provides a polypeptide which:

a) has lipolytic enzyme activity, and

b) has an amino acid sequence which has at least 80% identity (particularly at least 90% or at least 95% identity) to CALB (SEQ ID NO: 1) and has a difference from CALB (SEQ ID NO: 1) which comprises an amino acid substitution, deletion or insertion at a position corresponding to any of residues 1, 13, 25, 38-51, 53-55, 58, 69-79, 91, 92, 96, 97, 99, 103, 104-110, 113, 132-168, 173, 187-193, 197-205, 215, 223-231, 242, 244, 256, 259, 261-298, 303, 305, 308-313, or 315.

Finally, the invention provides use of the above variant polypeptide in a lipase-catalyzed process.

### BRIEF DESCRIPTION OF THE DRAWINGS

FIG. 1 shows an alignment of amino acid sequences SEQ ID NOS. 1-7.

### DETAILED DESCRIPTION OF THE INVENTION

### Parent Polypeptide

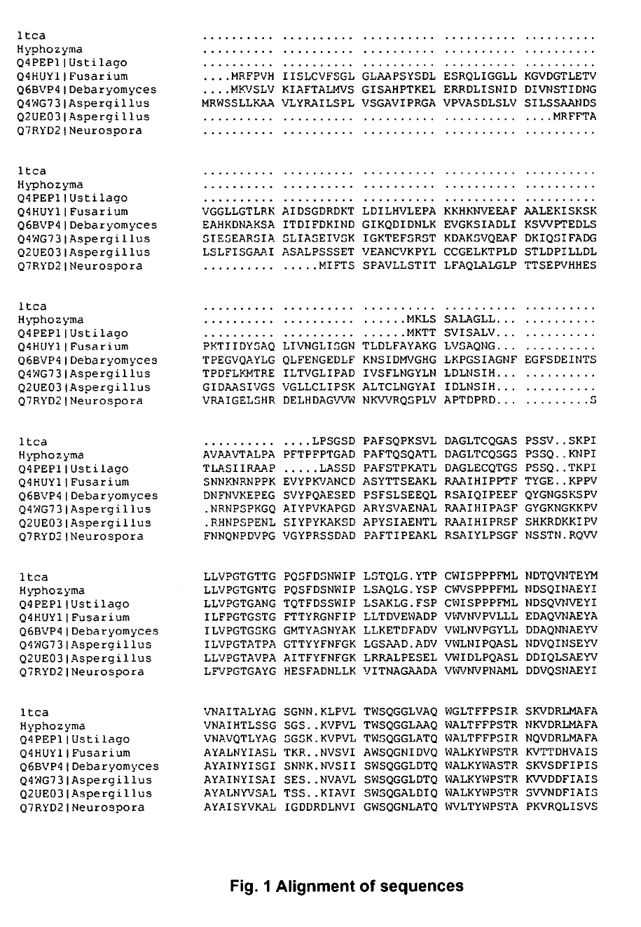

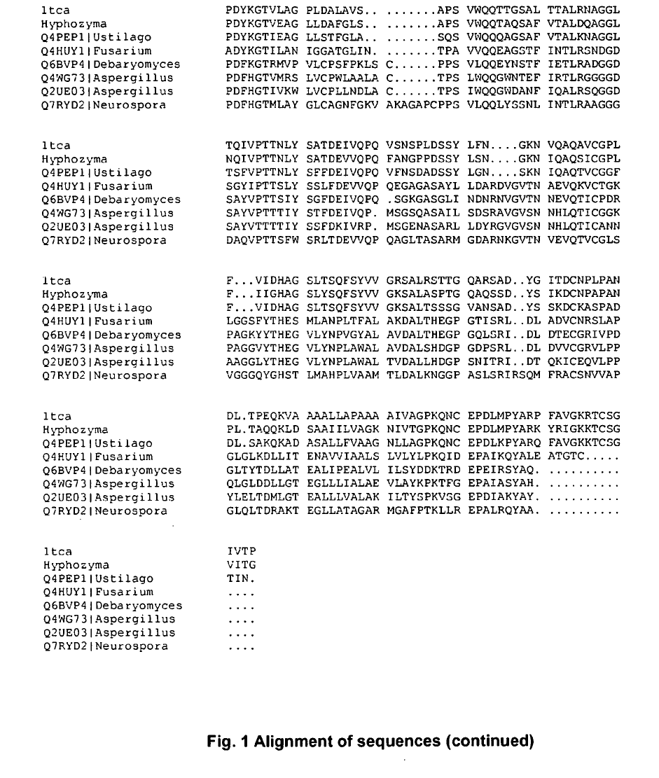

The parent polypeptide has lipolytic enzyme activity and has an amino acid sequence with at least 30% identity (particularly at least 40%, at least 50%, at least 60%, at least 70%, at least 80% or at least 90%) to Candida antarctica lipase B (CALB, SEQ ID NO: 1) which is described in WO8802775, and whose sequence is given in Uppenberg, J., Hansen, M. T., Patkar, S., Jones, T. A., Structure v2 pp. 293-308, 1994. The parent polypeptide may be any of the following lipases. An alignment is shown in FIG. 1.

SEQ ID NO: 1: Candida antarctica lipase B (CALB), 1TCA

SEQ ID NO: 2: Hyphozyma sp., WO9324619

SEQ ID NO: 3: Ustilago maydis, UniProt Q4pep1

SEQ ID NO: 4: Gibberella zeae (Fusarium graminearum), UniProt Q4HUY1

SEQ ID NO: 5: Debaryomyces hansenii, UniProt Q6BVP4

SEQ ID NO: 6: Aspergillus fumigatus, UniProt Q4WG73

SEQ ID NO: 7: Aspergillus oryzae, UniProt Q2UE03

SEQ ID NO: 8: Neurospora crassa lipase, UniProt Q7RYD2

The alignment was done using the needle program from the EMBOSS package (http://www.emboss.org) version 2.8.0 with the following parameters: Gap opening penalty: 10.00, Gap extension penalty: 0.50, Substitution matrix: EBLOSUM62. The software is described in EMBOSS: The European Molecular Biology Open Software Suite (2000), Rice, P. Longden, I. and Bleasby, A., Trends in Genetics 16, (6) pp 276-277. The program needle implements the global alignment algorithm described in Needleman, S. B. and Wunsch, C. D. (1970) J. Mol. Biol. 48, 443-453, and Kruskal, J. B. (1983).

Other parent polypeptides may aligned to the sequences in FIG. 1 by the same method or by the methods described in D. Sankoff and J. B. Kruskal, (ed.), Time warps, string edits and macromolecules: the theory and practice of sequence comparison, pp. 1-44 Addison Wesley.

### Three-Dimensional (3D) Structure and Lids

In the 3D structure 1TCA, the inventors identified two lids with high mobility at amino acid residues from 135 or 136 to 155 or 160 (Lid 1) and residues 267-295 (Lid 2) of SEQ ID NO: 1. The MD simulation indicated that the following regions are of particular interest because of a particularly high mobility: residues 141-149 in Lid 1 and the following regions in Lid 2: 267-269, 272, 275-276, 279-280, 282-283, 286-290.

### Selection of Amino Acid Residue

An amino acid residue may be selected having a non-hydrogen atom within 8 Å of a non-hydrogen atom of a residue in Lid 1 or Lid 2 in a 3D structure. This criterion selects the following residues in the structure 1TCA: 38-51, 53-55, 58, 69-79, 104-110, 113, 132-168, 173, 187-193, 197-205, 223-231, 259, 261-298, 305, 308-313, 315 of SEQ ID NO: 1.

The residue may particularly be selected within 6 A of the lids, leading to the following residues in 1TCA: 40-42, 46-51, 54, 58, 70-77, 79, 104-107, 109, 133-165, 167, 173, 187-192, 197-203, 223-225, 228-229, 261-297, 308-312.

An amino acid residue may also be selected by aligning homologous lipolytic enzyme sequences and selecting a residue at a position with variability, i.e. a position where different sequences have different residues. Thus, the following residues in CALB (SEQ ID NO: 1) can be selected by a comparison with Hyphozyma lipase (SEQ ID NO: 2) based on the alignment shown in FIG. 1: 1, 3, 5, 10, 12-15, 25, 30, 31, 32, 57, 62, 66, 76, 78, 80, 83, 88, 89, 91, 92, 96, 97, 114, 121, 123, 143, 147-149, 159, 163, 164, 168, 169, 174, 188, 194, 195, 197, 199, 205, 210, 214, 215, 221, 223, 229, 238, 242, 244, 249, 251, 254, 256, 261, 265, 268, 269, 272-274, 277-280, 282-284, 287, 303-306, 309, 314, 315, 317.

The following residues are of special interest: 1, 13, 25, 38, 42, 74, 140, 143, 147, 164, 168, 190, 199, 215, 223, 242, 244, 256, 265, 277, 280, 281, 283, 284, 285, 292, 303, 315 of CALB (SEQ ID NO: 1).

Corresponding residues in other lipases may be identified from a sequence alignment. An alignment of several sequences is shown in FIG. 1. Other sequences may be aligned by known methods, such as AlignX (a component of vector nti suite 9.0.0) using standard settings.

### Altered Amino Acid Sequence

The altered amino acid sequence is derived from the parent sequence by making an amino acid alteration at one or more selected positions, and optionally also at other positions. Each amino acid alteration consists of substitution or deletion of the selected residue or insertion of at least one residue adjacent to the selected residue at the N- or C-terminal side.

### Particular Substitutions

The following alterations in SEQ ID NO: 1 may optionally be combined:
K13Q, A25G, P38V,L,S, T42N, N74Q, V78I, Y91S, A92S, N96S, L99V, W104H, D134L,M,N, T138L, L140E, P143S,L, D145S, A146T, L147N,F, A148P, V149P, S150A, W155Q,N, Q157N, T158S, L163F, T164V, R168D, V190,IA, S197L,G, L199P, V215I, D223G, T229Y, R242A, T244P, T256K, L261A, D265P, P268A, E269Q, L277I, P280V, A281S, A283K, A284N, 1285E,D, G288D, N292C,Q, P303K, K308D, ot V315I.
Multiple substitutions:
   1258D G288D
   S197G L199P
   T164V L163F
   V190X Q157X
   A281X W155X
   D223X A281X
   D223X I285X
   A281X I285X
   A281X W155X A148X
   D145X K308X K138X
   D223X A281X1285X
Insertions: L147FN, G137ASV, V190GAH, L1QL, L1QGPL
Deletion: N97*

Based on an alignment such as that shown in FIG. 1, one sequence may be used as a template for alterations in another sequence. Thus, Lid 1 or Lid 2 of one sequence may be substituted with the corresponding lid region of another sequence. The following variants are designed by altering Lid 1 of CALB using the indicated polypeptide as template:
Q7RYD2 (Neurospora crassa) as termplate: Y135F K136H V139M G142Y P143G D145C L147G A148N V149F S150GKVAKAGAPC A151P W155L
Q4HUY1 (Fusarium graminearum) as template: V139I G142N P143I L144G D145G L147T A148G V149L S150IN A151T S153A W155V
Hyphozyma sp. lipase as template: L140E P143L L147F A148G V149L.
Q4PEP1 (Ustilago maydis) as template: V139I L140E P143L D145S A146T L147F A148G V149L S150A A151S P152Q.

Each of the above variants may optionally be combined with N292C and/or D223G and/or A281S and/or 1285E.

The following substitutions may be made in SEQ ID NO: 2 (Hyphozyma sp. lipase): V192I, Q159N, D136L,M,N, P41V,L, S50A, N45S, W106H.

### Nomenclature for Amino Acid Alterations

In this specification, an amino acid substitution is described by use of one-letter codes, e.g. W155Q. X is used to indicate a substitution with any different residue (e.g. V190X). Multiple substitutions are concatenated, e.g. S197G L199P to indicate a variant with two substitutions. Alternatives are indicated by commas, e.g. W155Q,N to indicate a substitution of W155 with Q or N. An asterisk indicates a deletion. An insertion is indicated as substitution of one residue with two or more residues (e.g. L147FN)

### Lipolytic Enzyme Activity

The parent and the variant polypeptides have lipolytic enzyme activity (particularly lipase activity), i.e. they are able to hydrolyze carboxylic ester bonds to release carboxylate (EC 3.1.1), particularly ester bonds in triglycerides (triacylglycerol lipase activity, EC 3.1.1.3).

The enzyme activity may be expressed as specific activity, i.e. hydrolytic activity per mg of enzyme protein. The amount of enzyme protein can be determined e.g. from absorption at 280 nm or by active-site titration (AST), as described by Rotticci et al. Biochim. Biophys. Acta 2000, 1483, 132-140.

### Enantioselectivity

Enantioselectivity is often an important parameter in CaLB catalyzed reactions, both in the hydrolysis and in the synthesis direction. The substrate can be a racemic mixture of two enantiomers, or it can be a prochiral meso form. In both cases a single enantiomer product is often desired. Enantiomeric excess (ee) is measured by quantifying the amount of both product enantiomers, and then calculating ee=(yield of desired enantiomer−yield of other enantiomer)/(sum of both yields). The quantification is often by chiral gas chromatography (GC) or high-performance liquid chromatography (HPLC).

### Use of Lipolytic Enzyme Variant

The lipolytic enzyme variant may be used for biocatalysis in a lipase-catalyzed reaction, both in ester hydrolysis and synthesis reactions, e.g. in synthesis of some polymers. The lipase-catalyzed reaction may be:

a) hydrolysis with a carboxylic acid ester and water as reactants, and a free carboxylic acid and an alcohol as products,

b) ester synthesis with a free carboxylic acid and an alcohol as reactants, and a carboxylic acid ester as product,

c) alcoholysis with a carboxylic acid ester and an alcohol as reactants, or

d) acidolysis with a carboxylic acid ester and a free fatty acid as reactants.

Like CALB, the variant of the invention may particularly be used in applications where the enzyme's chemo-, regio-, and/or stereoselectivity, stability and reaction rate or the ability to accept a relatively broad range of substrates is important. The reaction products are typically used in the chemical, fine chemical, pharmaceutical, or agrochemical industry, or as food ingredients. The variant may be immobilized, e.g. by adsorption on an adsorbent resin such as polypropylene.

The ester in the lipase-catalyzed reaction may have a bulky acid group or a bulky or secondary alcohol part, such as pNP 2-Me-butyrate, 6,8-difluro-4-methylumbelliferyl octanoate (DiFMU octanoate) or an iso-propyl fatty acid ester (e.g. C₁₆-C₁₈ fatty acid which may be saturated or unsaturated).

The variant may be used as described for CALB in A. J. J. Straathof, S. Panke, A. Schmid. Curr. Opin. Biotechnol. 2002, 13, 548-556; E. M. Anderson, K. M. Larsson, O. Kirk. Biocat. Biotrans. 1998, 16, 181-204; R. A. Gross, A. Kumar, B. Kaira. Chem. Rev. 2001, 101, 2097-2124).

### Examples

### Example 1

### Selection of Amino Acid Residues by Molecular Dynamics

From Molecular Dynamics simulations 2 regions were found to be of high importance for the activity of Candida antarctica lipase B, as follows.

CHARMm was used to prepare the 1TCA structure for the simulations. Hydrogen atoms were added to both protein and waters using the command HBUILD. The system was embedded in explicit water molecules and confined to a cubic box of side equal to 90 Angstroms. There were in total 24630 water molecules including those already present in the 1TCA structure. A simulation at constant temperature, 300K, and constant pressure, 1.01325 atmospheres, was performed for a total of 20 nanoseconds using NAMD. Berendsen's coupling method was used to keep the temperature and the pressure at the desired values. The results of the simulation were then analyzed using CHARMm (References for CHARMM: MacKerell, A. D., Bashford, D., Bellott, M., Dunbrack, R. L., Evanseck, J. D., Field, M. J., Fischer, S., Gao, J., Guo, H., Ha, S., Joseph-McCarthy, D., Kuchnir, L., Kuczera, K., Lau, F. T. K., Mattos, C., Michnick, S., Ngo, T., Nguyen, D. T., Prodhom, B., Reiher, W. E., Roux, B., Schlenkrich, M., Smith, J. C., Stote, R., Straub, J., Watanabe, M., Wiorkiewicz-Kuczera, J., Yin, D., Karplus, M. J. Phys. Chem. B 1998, 102, 3586; MacKerell, A. D., Jr., Brooks, B., Brooks, C. L., III, Nilsson, L., Roux, B., Won, Y., Karplus, M. In The Encyclopedia of Computational Chemistry; Schleyer, P. v. R. et al., Eds.; John Wiley & Sons: Chichester, 1998; Vol. 1, p 271; Brooks, B. R., Bruccoleri, R. E., Olafson, B. D., States, D. J., Swaminathan, S., Karplus, M. J. Comput. Chem. 1983, 4, 187).

The analysis revealed hitherto unknown lids with high mobility. Several regions were found to move when the enzyme is in solution. It was concluded that the enzyme functionality and specificity are dependent on this mobility and the specific structure present in the media of choice for the hydrolysis or the synthesis reaction. The simulation indicated a more closed like form in water solution and a more fully open form in organic solvent solution, i.e. more like the crystal structure in some surfactant containing water solution.

Using calculation of the isotropic Root Mean Square Displacements for the C-alpha atoms of the residues in CALB along the above mentioned simulation, regions with increased mobility were identified. The mobile lid regions were found to be residues 136-160 for Lid1 and residues 267-295 for Lid2. It was concluded that the residues in the neighborhood of these novel lids interact with the lid mobility and are thus very important for the activity of the enzyme.

### Example 2

### Hydrolysis Reactions

Hydrolytic activity of the variants was evaluated on pNP-butyrate, racemic pNP 2-methylbutyrate, and 6,8-difluro-4-methylumbelliferyl octanoate (DiFMU octanoate). Racemic pNP 2-Me-butyrate was synthesized according to J. Biol. Chem. 1971, 246, 6019-6023. DiFMU octanoate, purchased from Molecular Probes, has previously been reported by Lutz et al. (J. Am. Chem. Soc. 2005, 127, 13466-13467) in CALB assays. Whereas pNP 2-Me-butyrate selects variants with improved acceptance of substrates with a bulky acid group, DiFMU octanoate selects variants with improved acceptance of a bulky alcohol part. Reactions were performed in 50 mM aqueous phosphate buffer, pH 7.0 with 0.1% Triton X-100. Reaction kinetic was followed for approx. 15 min in microtiter plates, measuring at 405 nm (pNP) or 350/485 nm (ex/em for DiFMU). Activities were normalized based on enzyme A₂₈₀.

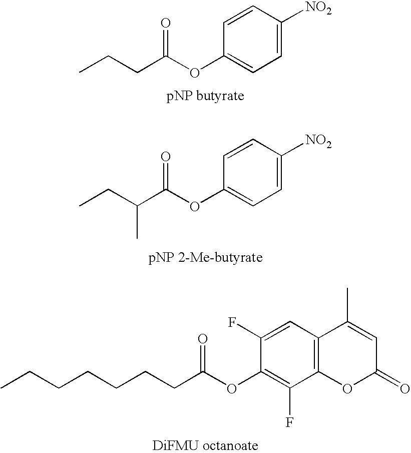

Results are shown below as activity for the various substrates in % of CALB wild-type.

| | pNP | pNP 2-Me- | DiFMU |
|---|---|---|---|
| Variant | butyrate | butyrate | octanoate |
| N74Q | 77 | 113 | 110 |
| P143S | 50 | 113 | 42 |
| A281S | 215 | 232 | 208 |
| P38S | 35 | 107 | 44 |
| N292Q | 73 | 158 | 105 |
| L1QGPL | 63 | 144 | 85 |
| L1QL | 65 | 193 | 49 |
| I285E | 233 | 332 | 236 |
| L147F | 98 | 232 | 90 |
| L147N | 79 | 178 | 79 |
| N292C | 80 | 282 | 90 |
| L140E | 51 | 151 | 79 |
| P143L | 79 | 192 | 112 |
| A146T | 55 | 126 | 42 |
| P280V | 48 | 100 | 36 |
| A283K | 104 | 115 | 94 |
| A284N | 65 | 125 | 19 |
| T103G, A148P | 70 | 167 | 0 |
| W104H, A148P | 11 | 146 | 0 |
| N74Q, A281S | 88 | 156 | 0 |
| V190A | 64 | 143 | |
| L199P | 74 | 162 | 75 |
| T256K | 105 | 120 | 79 |
| T42N | 35 | 216 | 47 |
| R242A | 24 | 119 | 39 |
| V215I | 105 | 133 | 43 |
| T164V | 75 | 130 | 80 |
| L163F, T164V | 81 | 160 | 92 |
| D265P | 28 | 117 | 44 |
| P303K | 35 | 108 | 50 |
| R168D | 62 | 122 | 53 |
| A25G | 66 | 111 | 26 |
| V315I | 65 | 102 | 19 |
| T244P | 56 | 146 | 20 |
| K13Q | 56 | 122 | 39 |
| L277I | 53 | 137 | 51 |
| Y91S, A92S, N96S, | 39 | 135 | 76 |
| N97*, L99V | | | |
| D223G | 830 | 3621 | 820 |
| Parent (CALB) | 100 | 100 | 100 |

The results demonstrate that the specific activity towards a bulky substrate (ester with a branched fatty acid) can be increased up to 37-fold by substituting a single selected amino acid residue.

### Example 3

### Variants with Lid Replacement

Variants based on CaLB wild-type (SEQ ID NO: 1) were designed by replacing lid 1 with the corresponding residues of the Fusarium lipase (SEQ ID NO: 4), the Debaryomyces lipase (SEQ ID NO: 5) or the Neurospora lipase (SEQ ID NO: 8). Further variants were designed by combining this with a single substitution of a selected residue (A281S). Results are expressed as activity in % of CALB activity on the same substrate.

| | | pNP | |
|---|---|---|---|
| | pNP | 2-Me- | DiFMU |
| Variant | butyrate | butyrate | octanoate |
| V139I, G142N, P143I, L144G, D145G, | 187 | 661 | 836 |
| L147T, A148G, V149L, S150IN, | | | |
| A151T, S153A, W155V | | | |
| Y135F, V139R, L140M, A141V, | 62 | 306 | 14 |
| G142P, P143V, D145C, A146P, | | | |
| L147S, A148F, V149P, S150KLSC, | | | |
| A151P, W155L | | | |
| Y135F, K136H, V139M, G142Y, | 341 | 1223 | 14 |
| P143G, D145C, L147G, A148N, | | | |
| V149F, S150GKVAKAGAPC, A151P, | | | |
| W155L | | | |
| V139I, G142N, P143I, L144G, D145G, | 1052 | 1612 | 631 |
| L147T, A148G, V149L, S150IN, | | | |
| A151T, S153A, W155V, A281S | | | |
| Y135F, K136H, V139M, G142Y, | 378 | 2397 | 76 |
| P143G, D145C, L147G, A148N, | | | |
| V149F, S150GKVAKAGAPC, A151P, | | | |
| W155L, A281S | | | |

The results demonstrate that the specific activity towards a bulky substrate can be significantly increased by replacing the lid of one lipase with the lid of another lipase, and that this can be further increased by combining with a single substitution of a selected residue.

### Example 4

### Enantioselectivity

Hydrolysis reactions were performed in 2 mL scale using 2 mM pNP 2-Me-butyrate as substrate in sodium phosphate buffer, 0.5 M pH 7.0 with 1% Triton X-100. The reactions were stopped by addition of 2 M HCl (0.1 mL), and then extracted into Et₂O (2 mL). After analysis by chiral GC (Varian CP-Chiralsil-DEX CB 10 m colum, temperature program 80 to 180° C. at 2° C./min), E (enantiomeric ratio) was calculated as E=ln[eeₚ(1−eeₛ)/(eeₚ+eeₛ)]/ln[eeₚ(1+eeₛ)/(eeₚ+eeₛ)], with eeₛ and eeₚ being ee (enantiomeric excess) for substrate and product, respectively. Reactions were performed in triplets for each enzyme (stopped at different conversions) and E reported as an average.

CALB was tested and compared with variant Y135F, K136H, V139M, G142Y, P143G, D145C, L147G, A148N, V149F, S150GKVAKAGAPC, A151P, W155L. The results were E=2.4 for the variant and E=1.05 for the parent lipase (CALB), showing that CALB is almost entirely non-selective, but the variant has an increased enantioselectivity.

### Example 5

### Hydrolysis of Long-Chain Fatty Acid Ester

Michaelis-Menten constants were determined for a CALB variant with pNP laurate as a long-chain substrate. Experiments were performed in 0.5 M sodium phosphate buffer, pH 7.0, containing 1% Triton X-100 (to avoid turbid solutions at high substrate concentrations).

| | | | k_{cat} /K_{M} |
|---|---|---|---|
| | k_{cat} (s⁻¹) | K_{M} (micro-M) | (s⁻¹ M⁻¹) |
| Parent (caLB) | 3.1 | 535 | 0.58 * 10⁴ |
| V139I, G142N, P143I, L144G, | 23 | 170 | 14 * 10⁴ |
| D145G, L147T, A148G, V149L, | | | |
| S150IN, A151T, S153A, W155V | | | |

The results show that the variant is 23 times more active than the parent lipase on the long-chain substrate (measured as k_{cal}/K_{M}).

### Example 6

### Hydrolysis of Iso-Propyl Ester

The variant used in the previous example was also tested in hydrolysis of iso-propyl palmitate. The results showed that the hydrolysis was 26% higher for the variant than for CALB. The hydrolysis was performed as follows:

As substrate, isopropylpalmitate was added to a concentration of 3 mg/ml in 50 mM NaAcetate pH 5.0 (=buffer), heated to 60° C. for 5 minutes and homogenized by Ultra Turrax for 45 seconds and used immediately after preparation. Purified enzyme preparations were diluted to a concentration corresponding to OD280=0.00016 in desalted water and 10 ppm Triton X-100. In PCR-plates 20 micro-L buffer, 60 micro-L substrate and 20 micro-L enzyme solution were mixed at 800 RPM for 20 seconds and transferred to a PCR thermocycler for 30 minutes reaction at 30 C followed by 5 minutes at 90° C. to inactivate enzymes and addition of 20 micro-L 10% solution of TritonX100 (in desalted water). The amount for fatty acids produced was determined using the NEFA C kit from Wako and results were calculated as an average of 6 determinations and subtraction of enzyme blank.

### Example 7

### Activity at High pH

Lipase activity of two CALB variants was measured at various pH at 30° C. with tributyrin as substrate and gum arabic as emulsifier. The results are expressed as relative activity, taking activity at pH 7.0 as 100.

| | pH 5.0 | pH 6.0 | pH 7.0 | pH 8.0 | pH 9.0 |
|---|---|---|---|---|---|
| Y135F, K136H, V139M, | 53 | 97 | 100 | 90 | 151 |
| G142Y, P143G, D145C, | | | | | |
| L147G, A148N, V149F, | | | | | |
| S150GKVAKAGAPC, | | | | | |
| A151P, W155L | | | | | |
| V139I, G142N, P143I, | 41 | 76 | 100 | 99 | 148 |
| L144G, D145G, L147T, | | | | | |
| A148G, V149L, S150IN, | | | | | |
| A151T, S153A, W155V | | | | | |
| Parent lipase (CALB) | 47 | 62 | 100 | 60 | 49 |

The variants are seen to have increased activity at alkaline pH (pH 7-9) and a higher pH optimum.

### Example 8

### Synthesis Reactions

The variants were immobilized on Accurel porous polypropylene by physical adsorption to a loading of 20 mg/g (based on A280). Reactions were performed in Eppendorf tubes with 1 mmol of each reagent, approx. 0.8 mL hexane, and 5 mg immobilized enzyme @ 40° C., 1200 rpm. Samples were withdrawn for analysis by NMR and chiral GC.

Results from a synthesis reaction with 2-ethyl-1-hexanol and vinyl acetate as reactants are shown below as conversion % (ee %):

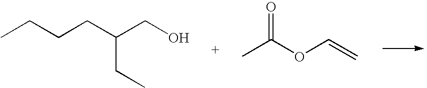

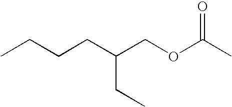

| Variant | 15 min | 30 min | 1 h | 2 h | 3 h |
|---|---|---|---|---|---|
| Parent (CaLB) | 11 (32) | 24 (27) | 43 (22) | 61 (17) | 63 (17) |
| Y135F K136H V139M | 6 (46) | 13 (46) | 24 (45) | 41 (40) | 52 (38) |
| G142Y P143G D145C | | | | | |
| L147G A148N V149F | | | | | |
| S150GKVAKAGAPC | | | | | |
| A151P W155L | | | | | |
| V139I G142N P143I | 0.1 (51) | 6 (49) | 12 (50) | 22 (49) | 33 (48) |
| L144G D145G L147T | | | | | |
| A148G V149L S150IN | | | | | |
| A151T S153A W155V | | | | | |

The enantiomeric ratio was calculated by the formula given above. The results were E=1.9 for the parent lipase (CALB), and E=3.0 and E=3.2 for the two variants. Thus, the results show improved enantioselectivity for the two variants.

Another experiment was made in the same manner, but with vinyl benzoate and 1-hexanol as reactants.

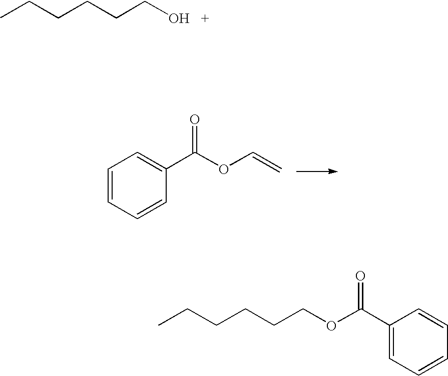

After 72 hours, a conversion of 17% was found for the variant 1285E, whereas the parent CALB gave 9%.

1-10. (canceled)

11. A method of preparing a polypeptide, comprising
a) selecting a parent polypeptide which has lipolytic enzyme activity and has an amino acid sequence with at least 30% identity to SEQ ID NO: 1,
b) selecting at least one amino acid residue in the sequence corresponding to any of residues 1, 13, 25, 38-51, 53-55, 58, 69-79, 91, 92, 96, 97, 99, 103, 104-110, 113, 132-168, 173, 187-193, 197-205, 215, 223-231, 242, 244, 256, 259, 261-298, 303, 305, 308-313, or 315 of SEQ ID NO: 1,
c) altering the amino acid sequence wherein the alteration comprises substitution or deletion of the selected residue or insertion of at least one residue adjacent to the selected residue,
d) preparing an altered polypeptide having the altered amino acid sequence,
e) determining the specific lipolytic enzyme activity, the lipolytic activity at alkaline pH and/or the enantioselectivity of the altered polypeptide, and
f) selecting an altered polypeptide which has a higher specific lipolytic enzyme activity, a higher activity at alkaline pH and/or an increased enantioselectivity than the parent polypeptide.

12. The method of claim 11 wherein the selected residue corresponds to any of residues 1, 13, 25, 38, 42, 74, 140, 143, 147, 164, 168, 190, 199, 215, 223, 242, 244, 256, 265, 277, 280, 281, 283, 284, 285, 292, 303, 315, 135-160 or 267-295 of SEQ ID NO: 1.

13. The method of claim 11 wherein the alteration comprises substitution of the selected residue with a residue found at the corresponding position of any of SEQ ID NOS: 1-8.

14. The method of claim 11 wherein the parent polypeptide is selected among SEQ ID NOS: 1-8.

15. The method of claim 11 wherein the parent polypeptide has an amino acid sequence with at least 90% identity to SEQ ID NO: 1.

16. A polypeptide which:
a) has lipolytic enzyme activity, and
b) has an amino acid sequence which has at least 80% identity to SEQ ID NO: 1 and compared to SEQ ID NO: 1 comprises an amino acid substitution, deletion or insertion at a position corresponding to any of residues 1, 13, 25, 38, 42, 74, 140, 143, 147, 164, 168, 190, 199, 215, 223, 242, 244, 256, 265, 277, 280, 283, 284, 285, 292, 303, 315, 135-160 or 267-295.

17. The polypeptide of claim 16 comprising an alteration corresponding to N74Q, P143S, A281S, P38S, N292Q, L1QGPL, L1QL, I285E, L147F, L147N, N292C, L140E, P143L, A146T, P280V, A283K, A284N, T103G, A148P, W104H, A148P, N74Q, A281S, V190A, L199P, T256K, T42N, R242A, V215I, T164V, L163F, T164V, D265P, P303K, R168D, A25G, V315I, T244P, K13Q, L277I, Y91S, A92S, N96S, N97*, L99V, or D223G.

18. The polypeptide of claim 16 which comprises a set of amino acid alterations compared to SEQ ID NO: 1 which is:
a) V139I G142N P143I L144G D145G L147TA148GV149LS150IN A151T S153A W155V;
b) Y135F V139R L140M A141V G142P P143V D145C A146P L147S A148F V149P S150KLSC A151P W155L;
c) Y135F K136H V139M G142Y P143G D145C L147G A148N V149F S150GKVAKAGAPC A151P W155L;
d) V139I G142N P143I L144G D145G L147T A148G V149L S150IN A151T S153A W155V A281S, or
e) Y135F K136H V139M G142Y P143G D145C L147G A148N V149F S150GKVAKAGAPC A151P W155L A281S.

19. The polypeptide of claim 16 which has an amino acid sequence which has at least 90% identity to SEQ ID NO: 1.

20. The polypeptide of claim 16 which has an amino acid sequence which has at least 95% identity to SEQ ID NO: 1.

21. The polypeptide of claim 16 in immobilized form.

22. A method of performing a lipase-catalyzed reaction, which comprises contacting a reactant with the polypeptide of claim 16 wherein the reaction is:
a) hydrolysis with a carboxylic acid ester and water as reactants, and a free carboxylic acid and an alcohol as products,
b) ester synthesis with a free carboxylic acid and an alcohol as reactants, and a carboxylic acid ester as product,
c) alcoholysis with a carboxylic acid ester and an alcohol as reactants, or
d) acidolysis with a carboxylic acid ester and a free fatty acid as reactants.

23. The method of claim 22, wherein the reaction is hydrolysis of an iso-propyl ester, or ester synthesis or alcoholysis with iso-propanol as a reactant.


SEQUENCE LISTING (8 sequences)

SEQ ID NO: 1 - protein, 317 aa, Candida antarctica [sequence omitted]

SEQ ID NO: 2 - protein, 319 aa, Hyphozyma species.
FTPFPTGADPAFTQSQATLDAGLTCQSGSPSSQKNPILLVPGTGNTGPQSFDSNWIPLSAQLGYSPCWVSPPPFMLNDSQINAEYIVNAIHTLSSGSGSKVPVLTWSQGGLAAQWALTFFPSTRNKVDRLMAFAPDYKGTVEAGLLDAFGLSAPSVWQQTAQSAFVTALDQAGGLNQIVPTTNLYSATDEVVQPQFANGPPDSSYLSNGKNIQAQSICGPLFIIGHAGSLYSQFSYVVGKSALASPTGQAQSSDYSIKDCNPAPANPLTAQQKLDSAAIILVAGKNIVTGPKQNCEPDLMPYARKYRIGKKTCSGVITG

SEQ ID NO: 3 - protein, 336 aa, Ustilago maydis
MKTTSVISALVTLASIIRAAPLASSDPAFSTPKATLDAGLECQTGSPSSQTKPILLVPGTGANGTQTFDSSWIPLSAKLGFSPCWISPPPFMLNDSQVNVEYIVNAVQTLYAGSGSKKVPVLTWSQGGLATQWALTFFPSIRNQVDRLMAFAPDYKGTIEAGLLSTFGLASQSVWQQQAGSAFVTALKNAGGLTSFVPTTNLYSFFDEIVQPQVFNSDADSSYLGNSKNIQAQTVCGGFFVIDHAGSLTSQFSYVVGKSALTSSSGVANSADYSSKDCKASPADDLSAKQKADASALLFVAAGNLLAGPKQNCEPDLKPYARQFAVGKKTCSGTIN

SEQ ID NO: 4 - protein, 445 aa, Gibberella zeae
APSYSDLESRQLIGGLLKGVDGTLETVVGGLLGTLRKAIDSGDRDKTLDILHVLEPAKKHKNVEEAFAALEKISKSKPKTIIDYSAQLIVNGLISGNTLDLFAYAKGLVSAQNGSNNKNRNPPKEVYPKVANCDASYTTSEAKLRAAIHIPPTFTYGEKPPVILFPGTGSTGFTTYRGNFIPLLTDVEWADPVWVNVPVLLLEDAQVNAEYAAYALNYIASLTKRNVSVIAWSQGNIDVQWALKYWPSTRKVTTDHVAISADYKGTILANIGGATGLINTPAVVQQEAGSTFINTLRSNDGDSGYIPTTSLYSSLFDEVVQPQEGAGASAYLLDARDVGVTNAEVQKVCTGKLGGSFYTHESMLANPLTFALAKDALTHEGPGTISRLDLADVCNRSLAPGLGLKDLLITENAVVIAALSLVLYLPKQIDEPAIKQYALEATGTC

SEQ ID NO: 5 - protein, 455 aa, Debaryomyces hansenii
HPTKELERRDLISNIDDIVNSTIDNGEAHKDNAKSAITDIFDKINDGIKQDIDNLKEVGKSIADLIKSVVPTEDLSTPEGVQAYLGQLFENGEDLFKNSIDMVGHGLKPGSIAGNFEGFSDEINTSDNFNVKEPEGSVYPQAESEDPSFSLSEEQLRSAIQIPEEFQYGNGSKSPVILVPGTGSKGGMTYASNYAKLLKETDFADVVWLNVPGYLLDDAQNNAEYVAYAINYISGISNNKNVSIISWSQGGLDTQWALKYWASTRSKVSDFIPISPDFKGTRMVPVLCPSFPKLSCPPSVLQQEYNSTFIETLRADGGDSAYVPTTSIYSGFDEIVQPQSGKGASGLINDNRNVGVTNNEVQTICPDRPAGKYYTHEGVLYNPVGYALAVDALTHEGPGQLSRIDLDTECGRIVPDGLTYTDLLATEALIPEALVLILSYDDKTRDEPEIRSYAQ

SEQ ID NO: 6 - protein, 440 aa, Aspergillus fumigatus
AVIPRGAVPVASDLSLVSILSSAANDSSIESEARSIASLIASEIVSKIGKTEFSRSTKDAKSVQEAFDKIQSIFADGTPDFLKMTREILTVGLIPADIVSFLNGYLNLDLNSIHNRNPSPKGQAIYPVKAPGDARYSVAENALRAAIHIPASFGYGKNGKKPVILVPGTATPAGTTYYFNFGKLGSAADADVVWLNIPQASLNDVQINSEYVAYAINYISAISESNVAVLSWSQGGLDTQWALKYWPSTRKVVDDFIAISPDFHGTVMRSLVCPWLAALACTPSLWQQGWNTEFIRTLRGGGGDSAYVPTTTIYSTFDEIVQPMSGSQASAILSDSRAVGVSNNHLQTICGGKPAGGVYTHEGVLYNPLAWALAVDALSHDGPGDPSRLDLDVVCGRVLPPQLGLDDLLGTEGLLLIALAEVLAYKPKTFGEPAIASYAH

SEQ ID NO: 7 - protein, 401 aa, Aspergillus oryzae
LPSSSETVEANCVKPYLCCGELKTPLDSTLDPILLDLGIDAASIVGSVGLLCLIPSKALTCLNGYAIIDLNSIHRHNPSPENLSIYPYKAKSDAPYSIAENTLRAAIHIPRSFSHKRDKKIPVLLVPGTAVPAAITFYFNFGKLRRALPESELVWIDLPQASLDDIQLSAEYVAYALNYVSALTSSKIAVISWSQGALDIQWALKYWPSTRSVVNDFIAISPDFHGTIVKWLVCPLLNDLACTPSIWQQGWDANFIQALRSQGGDSAYVTTTTIYSSFDKIVRPMSGENASARLLDYRGVGVSNNHLQTICANNAAGGLYTHEGVLYNPLAWALTVDALLHDGPSNITRIDTQKICEQVLPPYLELTDMLGTEALLLVALAKILTYSPKVSGEPDIAKYAY

SEQ ID NO: 8 - protein, 388 aa, Neurospora crassa
LPTTSEPVHHESVRAIGELSHRDELHDAGVVWNKVVRQSPLVAPTDPRDSFNNQNPDVPGVGYPRSSDADPAFTIPEAKLRSAIYLPSGFNSSTNRQVVLFVPGTGAYGHESFADNLLKVITNAGAADAVWVNVPNAMLDDVQSNAEYIAYAISYVKALIGDDRDLNVIGWSQGNLATQWVLTYWPSTAPKVRQLISVSPDFHGTMLAYGLCAGNFGKVAKAGAPCPPSVLQQLYSSNLINTLRAAGGGDAQVPTTSFWSRLTDEVVQPQAGLTASARMGDARNKGVTNVEVQTVCGLSVGGGQYGHSTLMAHPLVAAMTLDALKNGGPASLSRIRSQMFRACSNVVAPGLQLTDRAKTEGLLATAGARMGAFPTKLLREPALRQYAA